(19) United States
(12) Patent Application Publication
(10) Pub. No.: US 2025/0259697 A1
(43) Pub. Date: Aug. 14, 2025

(54) Single Panel Representation of Multiple Charge Evidence Linked to a Bond in the Protein

(71) Applicant: DH Technologies Development Pte. Ltd., Singapore (SG)

(72) Inventors: Stephen A. TATE, Barrie (CA); Claudia ALVAREZ, Vaughan (CA); Lyle Lorrence BURTON, Woodbridge (CA)

(21) Appl. No.: 18/856,349

(22) PCT Filed: Mar. 23, 2023
(86) PCT No.: PCT/IB2023/052880

§ 371 Date: Oct. 11, 2024

Related U.S. Application Data
(60) Provisional application No. 63/362,881, filed on Apr. 12, 2022.

(51) Int. Cl. G16B 15/20; G01N 33/68; G16B 40/10; G16B 45/00

(52) U.S. Cl. CPC G16B 15/20; G01N 33/6848; G16B 40/10; G16B 45/00

(57) ABSTRACT

A user interface is provided for displaying in the same panel and at the same time a sequence of a polymeric compound and multiple pieces of spectral evidence from an experimental product ion spectrum that are linked to a bond of the sequence. The sequence and the spectrum of the polymeric compound are received, where one or more product ions of the spectrum are assigned to at least one bond of the sequence. The sequence is displayed in a panel of a display device with at least one interactive icon between at least two elements of the sequence representing the bond. When the interactive icon is selected, at least two different spectral plots of the spectrum showing two different product ions of the spectrum that support a cleavage of the bond are displayed in the same panel of the sequence and at the same time as the sequence.

### RELATED APPLICATIONS

This application claims the benefit of U.S. Provisional Patent Application Ser. No. 63/362,881, filed on Apr. 12, 2022, the content of which is incorporated by reference herein in its entirety.

### FIELD

The teachings herein relate to providing a user interface for simultaneously displaying multiple pieces of spectral evidence linked to a bond of a polymeric compound.

### INTRODUCTION

### Manual Inspection Visualization Problem

Top or middle-down mass spectrometry experiments using electron-capture dissociation (ECD) fragmentation enable the determination of the correct sequence of amino acids in a protein. One issue commonly encountered from a top or middle-down experiment is that the product ion or MS/MS mass spectrum generated is very complex and thus not always straightforward to analyze. The complexity of the spectrum is the result of having many fragments which are inherently generated with ECD fragmentation. ECD fragmentation primarily produces two c′-(N-terminal) and z′-(C-terminal) product ions. These product ions can also exist as different charge states for each bond that is cleaved within the protein.

Conventional peak matching algorithms, such as the peak matching algorithm in ProteinPilot™ produced by Sciex of Framingham, MA, assign the spectrum based on the mass and isotopic distribution of the fragment. However, these peak matching algorithms can also identify many false positive matches or miss a peak selection due to having a high signal-to-noise threshold (false negative).

FIG. 2 is an exemplary zoomed-in plot 200 of a product ion mass spectrum showing a missed peak selection due to having a high signal to noise threshold, in accordance with various embodiments. Experimental product ion peaks, such as peak 210, that match theoretical isotopic peak pattern 220 are missed due to having the noise threshold too high.

FIG. 3 is an exemplary zoomed-in plot 300 of product ion mass spectrum showing a false positive due to an incorrect match with a theoretical isotopic peak pattern, in accordance with various embodiments. Experimental product ion peaks, such as peak 310, may be found to match theoretical isotopic peak pattern 320 by the peak matching algorithm. However, inspection of FIG. 3 quickly shows that the experimental peaks, including peak 310, do not fully match isotopic peak pattern 320. For this reason, manual inspection of individual spectra for each fragment and its charge states is often necessary to validate the matches selected by the peak picking algorithm.

Conventionally, a peak viewing tool can present the bond data as a list of product ions where the user selects one product ion, and its location in a spectrum is displayed. Alternatively, the bond data can be presented as a two-dimensional (2D) graphical map of charge state versus bond number where the user selects a bond and charge state, and the corresponding location in a spectrum is displayed.

FIG. 4 is an exemplary display panel 400 showing a list of product ions of bonds and a spectral plot of a zoomed-in section of the product ion spectrum corresponding to a product ion selected from the list, upon which embodiments of the present application may be implemented. In FIG. 4, single panel 410 initially displays subpanel 420. Subpanel 420 provides an interactive list of product ions found in an experimental spectrum for one or more bonds of a polymeric sequence. For each product ion, a plurality of additional information is presented including, but not limited to, the product ion type, the charge state, and the product ion sequence.

If a product ion of subpanel 420 is selected by a user, a single spectral plot of a zoomed-in section of the experimental spectrum, showing one or more spectral peaks representing the product ion, is displayed in single panel 410. For example, if the product ion of row 428 is selected, as shown in FIG. 4, then a spectral plot is shown in subpanel 430. The spectral plot of subpanel 430 shows a zoomed-in section of the experimental spectrum that includes one or more spectral peaks representing the selected product ion of row 428.

FIG. 5 is an exemplary display panel 500 showing bond data represented as a 2D graphical map of charge state versus bond number and a spectral plot of a zoomed-in section of the product ion spectrum corresponding to the bond and charge state selected from the 2D graphical map, upon which embodiments of the present application may be implemented. In FIG. 5, single panel 510 initially displays subpanel 520. Subpanel 520 provides an interactive 2D graphical map of charge state versus bond number. Shaded intersections of the rows and columns of the 2D map are product ions at different charge states that were putatively found in an experimental spectrum for one or bonds of a polymeric sequence. Each shaded product ion found can be selected by a user.

If a shaded product ion of subpanel 520 is selected by a user, a single spectral plot of a zoomed-in section of the experimental spectrum, showing one or more spectral peaks representing the product ion region, is displayed in single panel 510. For example, if shaded product ion 525 is selected, as shown in FIG. 5, then a spectral plot is shown in subpanel 530. The spectral plot of subpanel 530 shows a zoomed-in section of the experimental spectrum that includes one or more spectral peaks representing shaded product ion 525.

Unfortunately, however, for manual inspection of the data, the visualization method depicted in FIGS. 4 and 5 presents a challenge as the user needs to scroll up and down within the list or switch between panels to find the results that can support the cleavage of a bond. Ultimately, the issues listed above make the manual analysis of top or middle-down data harder for the user as it currently stands.

As a result, additional systems and methods are needed to visualize data from top or middle-down mass spectrometry experiments using ECD fragmentation during manual analysis of that data to reduce false negative and false positive product ion selections.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Fragmentation Techniques Background

Electron-based dissociation (ExD), ultraviolet photodissociation (UVPD), infrared photodissociation (IRMPD), and collision-induced dissociation (CID) are often used as fragmentation techniques for tandem mass spectrometry (MS/MS). CID is the most conventional technique for dissociation in tandem mass spectrometers.

ExD can include, but is not limited to, electron-induced dissociation (EID), electron impact excitation in organics (EIEIO), electron capture dissociation (ECD), or electron transfer dissociation (ETD).

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{AIL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 application”). In the '459 application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### SUMMARY

The teachings herein relate to providing a user interface for simultaneously displaying multiple pieces of spectral evidence linked to a bond of a polymeric compound. More particularly the teachings herein relate to systems and methods for displaying in the same viewing panel of a sequence of the polymeric compound and at the same time as the sequence at least two different spectral plots of a product ion spectrum showing two different product ion regions of the spectrum that support a cleavage of a bond of the sequence when an interactive icon is selected between two elements of the sequence.

The systems and methods herein can be performed in conjunction with a processor, controller, or computer system, such as the computer system of FIG. 1.

A system, method, and computer program product are disclosed for simultaneously displaying multiple pieces of spectral evidence linked to a bond of a polymeric compound. A sequence of a polymeric compound is received. At least one product ion spectrum of the polymeric compound is received. The one or more product ions of the at least one spectrum are assigned to at least one bond of the sequence.

The sequence is displayed in a panel of a display device with at least one interactive icon between at least two elements of the sequence representing the at least one bond. When the at least one interactive icon is selected, at least two different spectral plots of the at least one spectrum showing two different product ions of at least one spectrum that support a cleavage of the at least one bond are displayed in the same panel of the sequence and at the same time as the sequence.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary zoomed-in plot of a product ion mass spectrum showing a missed peak selection due to having a high signal to noise threshold, in accordance with various embodiments.

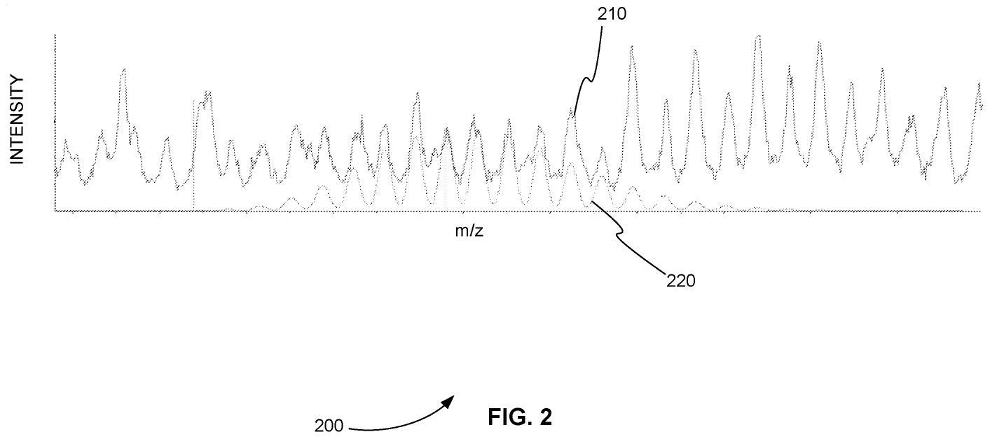

FIG. 3 is an exemplary zoomed-in plot of product ion mass spectrum showing a false positive due to an incorrect match with a theoretical isotopic peak pattern, in accordance with various embodiments.

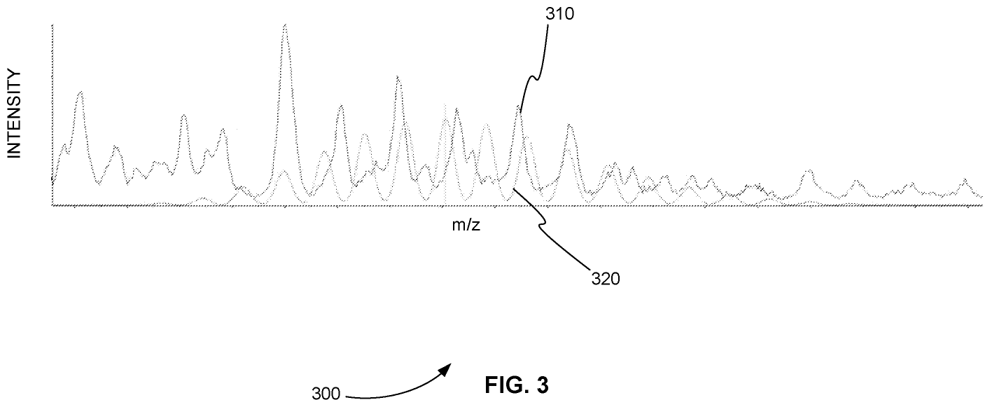

FIG. 4 is an exemplary display panel showing a list of product ions of bonds and a spectral plot of a zoomed-in section of the product ion spectrum corresponding to a product ion selected from the list, upon which embodiments of the present application may be implemented.

FIG. 5 is an exemplary display panel showing bond data represented as a 2D graphical map of charge state versus bond number and a spectral plot of a zoomed-in section of the product ion spectrum corresponding to the bond and charge state selected from the 2D graphical map, upon which embodiments of the present application may be implemented.

FIG. 6 is and exemplary user interface for simultaneously displaying multiple pieces of spectral evidence linked to a bond of a polymeric compound, in accordance with various embodiments.

FIG. 7 is a schematic diagram of a system for simultaneously displaying multiple pieces of spectral evidence linked to a bond of a polymeric compound, in accordance with various embodiments.

FIG. 8 is an exemplary flowchart showing a method for providing a user interface for simultaneously displaying multiple pieces of spectral evidence linked to a bond of a polymeric compound, in accordance with various embodiments.

FIG. 9 is a schematic diagram of a system that includes one or more distinct software modules and that performs a method for providing a user interface for simultaneously displaying multiple pieces of spectral evidence linked to a bond of a polymeric compound, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

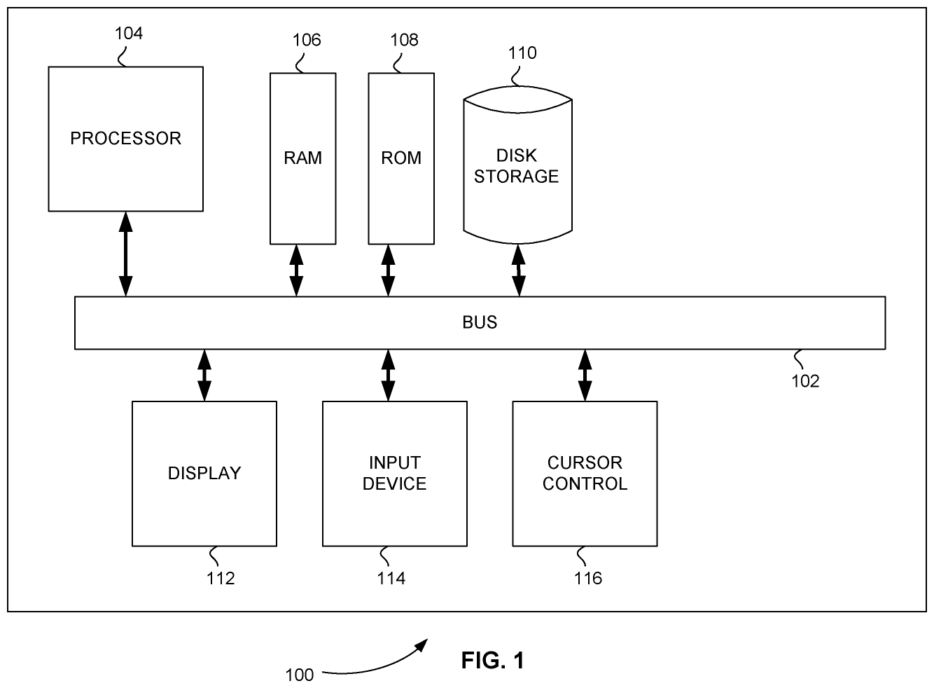

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed—to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### User Interface Displaying Multiple Bond Evidence

As described above, top or middle-down mass spectrometry experiments using ECD fragmentation enable the determination of the correct sequence of amino acids in a protein. One issue commonly encountered from a top or middle-down experiment is that the product ion mass spectrum generated is very complex and thus not always straightforward to analyze.

Conventional peak matching algorithms assign the spectrum based on the mass and isotopic distribution of the fragment. However, these peak matching algorithms can also identify many false positive matches or miss a peak selection due to having a high signal-to-noise threshold (false negative).

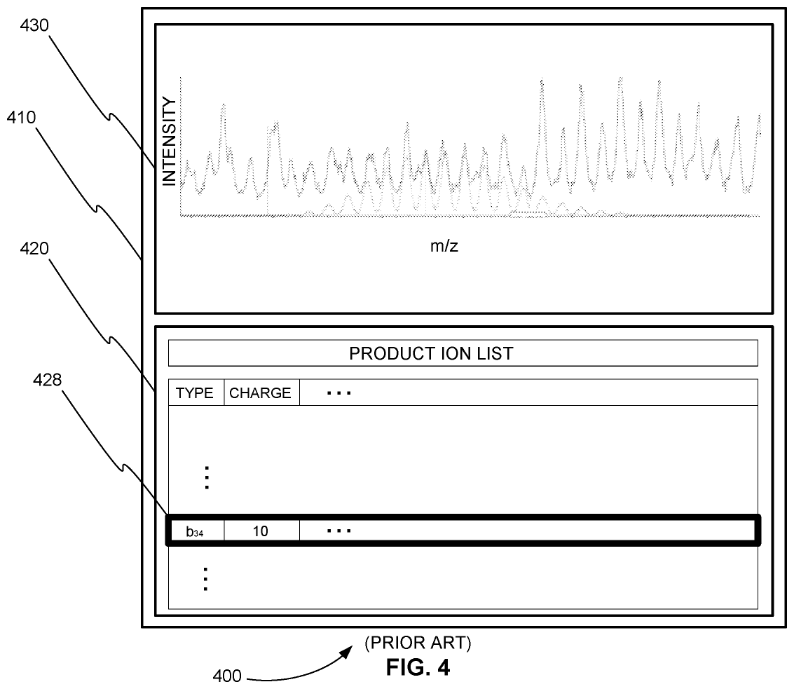

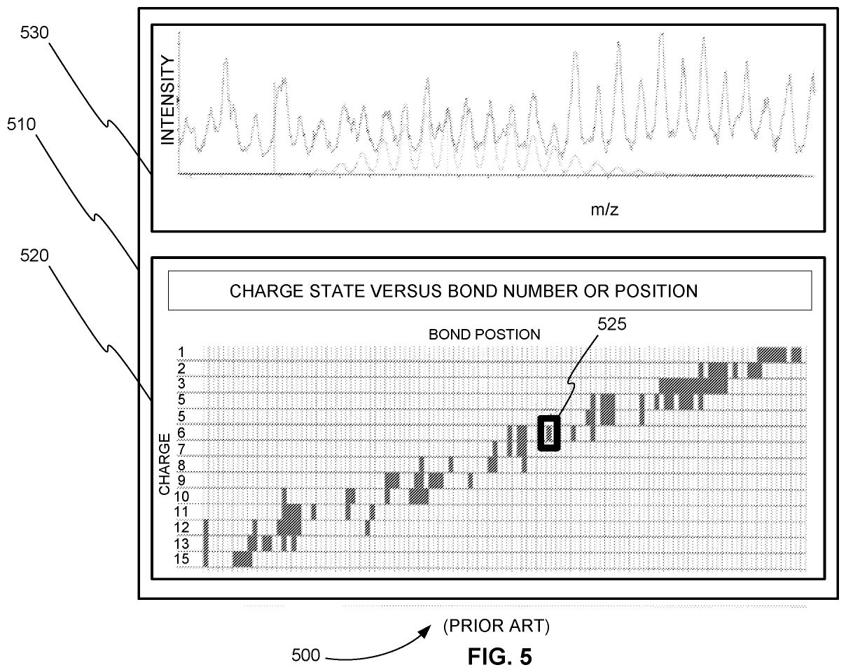

Conventionally, as shown in FIG. 4, a peak viewing tool presents the bond data as a list of product ions, where the user selects one product ion and its location in a spectrum is displayed. Alternatively, as shown in FIG. 5, the bond data can be presented as a 2D graphical map of charge state versus bond number, where the user selects a bond and charge state, and the corresponding location in a spectrum is displayed.

Unfortunately, however, for manual inspection of the data, the visualization method depicted in FIGS. 4 and 5 presents a challenge as the user needs to scroll up and down within the list or switch between panels to find the results that can support the cleavage of a bond. Ultimately, the issues listed above make the manual analysis of top or middle-down data harder for the user as it currently stands.

As a result, additional systems and methods are needed to visualize data from top or middle-down mass spectrometry experiments using ECD fragmentation during manual analysis of that data to reduce false negative and false positive product ion selections.

In various embodiments, a user interface is presented with a single panel view that provides multiple pieces of evidence of a bond cleavage at the same time for manual review and inspection of top or middle-down data. In this single pane, the full evidence for the cleavage of a protein bond resulting from ECD fragmentation is presented simultaneously. This panel is divided into different subpanels that enable the user to focus on a single bond of the protein sequence and analyze the different portions of a product ion spectrum that support the cleavage of that bond.

The portions of the product ion spectrum that are included in the panel can be those depicting c′-(N-terminal) and z′-(C-terminal) type product ions as well as their different charge states, for example, however other types of product ions can be shown also. Further, information about the parts per million (ppm) error, fragment sequences, and other features already present in conventional visualization panels, such as FIGS. 4 and 5, can also be included to provide more measures of confidence for the manual inspection. A ppm error or mass error is the difference between the theoretical mass-to-charge ratio (m/z) value of the theoretical product ion and the experimentally measured m/z value of the measured product ion.

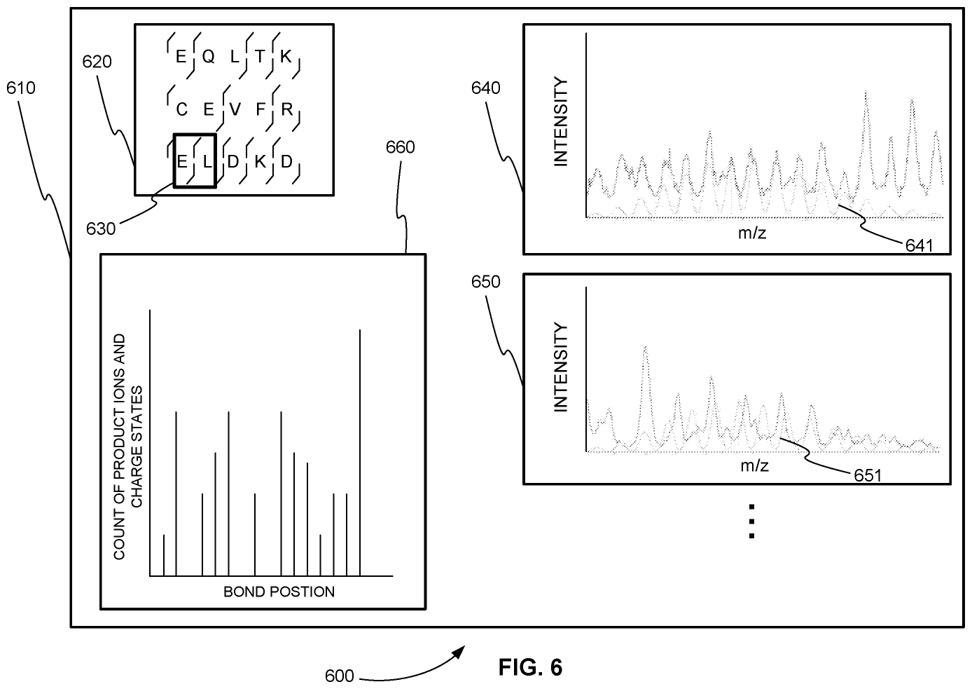

FIG. 6 is an exemplary user interface 600 for simultaneously displaying multiple pieces of spectral evidence linked to a bond of a polymeric compound, in accordance with various embodiments. Single panel 610 includes subpanel 620 for displaying a sequence of a polymeric compound. The polymeric compound can be a protein. In various alternative embodiments, the polymeric compound can be a ribonucleic acid (RNA) or a deoxyribonucleic acid (DNA), for example.

The sequence of subpanel 620 can include one or more interactive icons between sequence elements. In various embodiments, the presence of at least one interactive icon between sequence elements in subpanel 620 indicates that evidence was found for the bond between the two sequence elements. Similarly, in various embodiments, the absence of at least one interactive icon between sequence elements in subpanel 620 indicates that no evidence was found for the bond between the two sequence elements. Additionally, in various embodiments, the brightness or boldness of at least one interactive icon between sequence elements in subpanel 620 indicates the amount of evidence that was found for the bond between the two sequence elements. So, an interactive icon that is darker in color or bolder, for example, indicates more evidence for the bond. Color has only been previously used to depict the type of bond, for example. See https://www.ncbi.nlm.nih.gov/pmc/articles/PMC4445472/.

Upon selection of at least one interactive icon between elements in subpanel 620, all evidence for the corresponding bond is displayed at the same time in single panel 610. For example, as shown in FIG. 6, upon selection of at least one interactive icon in box 630, multiple zoomed-in spectral plots of the product ion spectrum are displayed at the same time in single panel 610. These multiple spectral plots show the product ion evidence for the bond corresponding to the at least one interactive icon in box 630.

Note that only two spectral plots are shown in subpanels 640 and 650 due to the size of the plots and the size of the viewable portion of single panel 610. In order to access multiple spectral plots extending to nonviewable portions of single panel 610, a scroll bar can be added, for example.

The user interface of FIG. 6 is an improvement for manual data analysis over the user interfaces shown in FIGS. 4 and 5 because it shows all of the evidence for bonds in one place and at the same time. By reviewing the data per cleaved bond, the user is not required to switch between panels or scroll longs lists to search for the results. In other words, this method of visualizing the data enables a user to more easily review and validate the results generated from ECD fragmentation.

In various embodiments, the spectral plots of single panel 610 further include theoretical product ion peaks or profiles. For example, the spectral plot of subpanel 640 includes theoretical product ion profile 641, and the spectral plot of subpanel 650 includes theoretical product ion profile 651.

In various embodiments, a count or score of product ions and charges states of the product ion mass spectrum that support a cleavage of a bond between each two elements of the sequence is calculated. This calculation produces a count or score of product ions and charge states per bond position.

In various embodiments, a plot of the count or score of product ions and charge states per bond position is also displayed in single panel 610. For example, subpanel 660 includes a plot of the count or score of product ions and charge states per bond position of the sequence shown in subpanel 620.

### System for Providing a User Interface

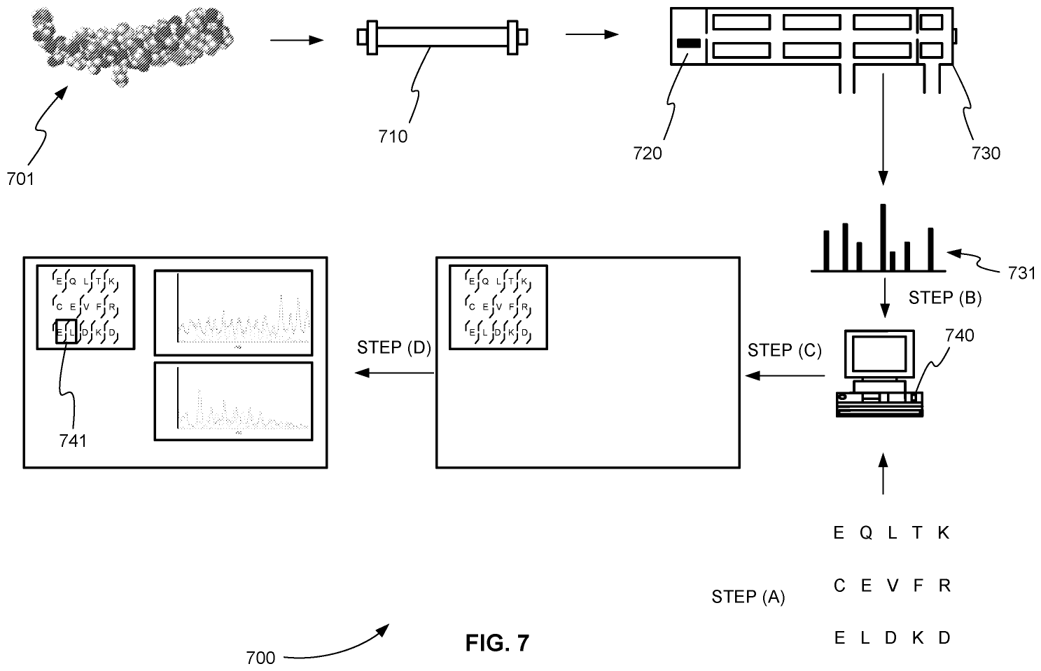

FIG. 7 is a schematic diagram 700 of a system for simultaneously displaying multiple pieces of spectral evidence linked to a bond of a polymeric compound, in accordance with various embodiments. The system includes processor 740. Processor 740 can be, but is not limited to, a controller, a computer, a microprocessor, the computer system of FIG. 1, or any device capable of analyzing data. Processor 740 can also be any device capable of sending and receiving control signals and data.

In a step (A), processor 740 receives a sequence of polymeric compound 701. The sequence is provided by a user, for example. A user, for example, knows the sequence of the protein and wants to confirm (or not) that the experimental data matches the sequence. In various embodiments, the sequence provided by a user may also contain one or more modifications to the sequence. The user may want to consider modifications, say particular amino acids are oxidized (which shifts the mass).

In a step (B), processor 740 receives at least one product ion spectrum 731 of polymeric compound 701. The one or more product ions of spectrum 731 are assigned to at least one bond of the sequence.

In step (C), processor 740 displays the sequence in a panel of a display device with interactive icon 741 between at least two elements of the sequence representing the at least one bond. In step (D), when interactive icon 741 is selected, processor 740 displays in the same panel of the sequence and at the same time as the sequence at least two different spectral plots of the at least one spectrum, showing two different product ions of at least one spectrum that support a cleavage of the at least one bond.

In various embodiments, spectrum 731 is produced using ECD.

In various embodiments, the sequence and the at least two different spectral plots are displayed in different subpanels of the same panel.

In various embodiments, the at least two different spectral plots are different zoomed-in portions of spectrum 731.

In various embodiments, the at least two different spectral plots further include theoretical product ion peaks or profiles of the two different product ions.

In various embodiments, the at least two different spectral plots further include a mass error of a product ion.

In various embodiments, the at least two different spectral plots further include a sequence of a product ion.

In various embodiments, the at least two different spectral plots further include different charge states of a product ion.

In various embodiments, the two different product ions include two complementary product ions of the sequence.

In various embodiments, the processor 740 further displays an interactive icon between each two elements of the sequence if a product ion of spectrum 731 is found to support a cleavage of a bond between the two elements.

In various embodiments, the processor 740 does not display an interactive icon between each two elements of the sequence if no product ions of spectrum 731 are found to support a cleavage of a bond between the two elements.

In various embodiments, the processor 740 further calculates a count or score of products ions of spectrum 731 that support a cleavage of a bond between each two elements of the sequence. A count or score or score of supporting product ions and charge states is produced per bond position.

In various embodiments, the processor 740 further displays an interactive icon between each two elements of the sequence that indicates the count or score or score if a product ion of spectrum 731 is found to support a cleavage of a bond between the two elements.

In various embodiments, the brightness of the color of the interactive icon indicates the count or score.

In various embodiments, the processor 740 further displays on the display device in the same panel a plot of the count or score per bond position of the sequence.

In various embodiments, the system of FIG. 7 further includes mass spectrometer 730 that measures mass spectrum 731 and sends mass spectrum 731 to processor 740. Ion source device 720 of mass spectrometer 730 ionizes separated fragments of compound 701 or only compound 701, producing an ion beam. Ion source device 720 is controlled by processor 740, for example. Ion source device 720 is shown as a component of mass spectrometer 730. In various alternative embodiments, ion source device 720 is a separate device. Ion source device 720 can be, but is not limited to, an electrospray ion source (ESI) device or a chemical ionization (CI) source device such as an atmospheric pressure chemical ionization source (APCI) device or an atmospheric pressure photoionization (APPI) source device.

Mass spectrometer 730 mass analyzes product ions of compound 701 or selects and fragments compound 701 and mass analyzes product ions of compound 701 from the ion beam at a plurality of different times. Mass spectrum 731 is produced for compound 701. Mass spectrometer 730 is controlled by processor 740, for example.

In the system of FIG. 7, mass spectrometer 730 is shown as a triple quadrupole device. One of ordinary skill in the art can appreciate that any component of mass spectrometer 730 can include other types of mass spectrometry devices including, but not limited to, ion traps, orbitraps, time-of-flight (TOF) devices, ion mobility devices, or Fourier transform ion cyclotron resonance (FT-ICR) devices.

In various embodiments, the system of FIG. 7 further includes additional device 710 that affects compound 701 providing the at least one additional dimension. As shown in FIG. 7, additional device 710 is an LC device and the at least one additional dimension or spectral data provided is retention time. In various alternative embodiments, additional device 710 can be, but is not limited to, a gas chromatography (GC) device, capillary electrophoresis (CE) device, an ion mobility spectrometry (IMS) device, or a differential mobility spectrometry (DMS) device. In still further embodiments, additional device 710 is not used and the at least one additional dimension or spectral data provided is precursor ion m/z and is provided by mass spectrometer 730 operating in a precursor ion scanning mode.

### Method for Providing a User Interface

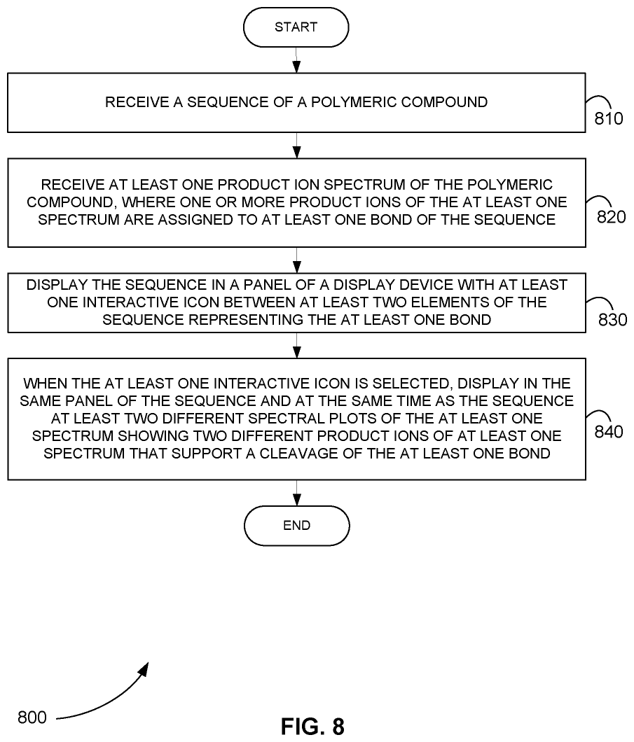

FIG. 8 is an exemplary flowchart showing a method 800 for providing a user interface for simultaneously displaying multiple pieces of spectral evidence linked to a bond of a polymeric compound, in accordance with various embodiments.

In step 810 of method 800, a sequence of a polymeric compound is received.

In step 820, at least one product ion spectrum of the polymeric compound is received. The one or more product ions of the at least one spectrum are assigned to at least one bond of the sequence.

In step 830, the sequence is displayed in a panel of a display device with at least one interactive icon between at least two elements of the sequence representing the at least one bond.

In step 840, when the at least one interactive icon is selected, at least two different spectral plots of the at least one spectrum showing two different product ions of at least one spectrum that support a cleavage of the at least one bond are displayed in the same panel of the sequence and at the same time as the sequence.

### Computer Program Product for Providing a User Interface

In various embodiments, a computer program product includes a non-transitory tangible computer-readable storage medium whose contents include a program with instructions being executed on a processor so as to perform a method for providing a user interface for simultaneously displaying multiple pieces of spectral evidence linked to a bond of a polymeric compound. This method is performed by a system that includes one or more distinct software modules.

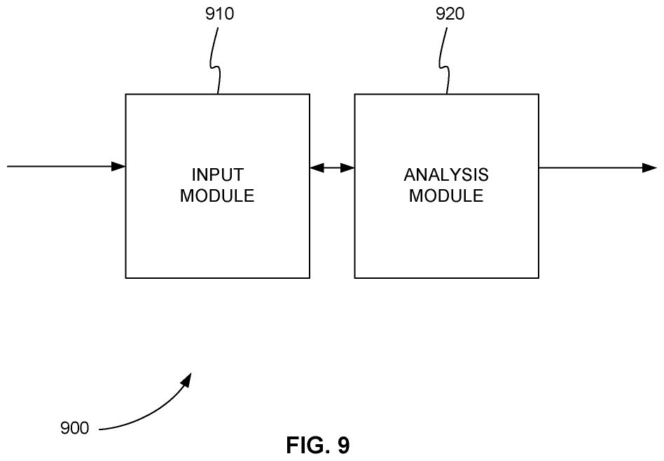

FIG. 9 is a schematic diagram of a system 900 that includes one or more distinct software modules and that performs a method for providing a user interface for simultaneously displaying multiple pieces of spectral evidence linked to a bond of a polymeric compound, in accordance with various embodiments. System 900 includes input module 910 and analysis module 920.

Input module 910 receives a polymeric compound sequence. Input module 910 receives at least one product ion spectrum known to include the polymeric compound. The one or more product ions of the at least one spectrum are assigned to at least one bond of the sequence.

Analysis module 910 displays the sequence in a panel of a display device with at least one interactive icon between at least two elements of the sequence representing a bond of the sequence.

When the at least one interactive icon is selected, analysis module 910 displays in the same panel of the sequence and at the same time as the sequence at least two different spectral plots showing two different product ions of the at least one spectrum that support a cleavage of the bond.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A method for providing a user interface for simultaneously displaying multiple pieces of spectral evidence linked to a bond of a polymeric compound, comprising:
(a) receiving a sequence of a polymeric compound;
(b) receiving at least one product ion spectrum of the polymeric compound, wherein one or more product ions of the at least one spectrum are assigned to at least one bond of the sequence;
(c) displaying the sequence in a panel of a display device with at least one interactive icon between at least two elements of the sequence representing the at least one bond; and
(d) when the at least one interactive icon is selected, displaying in the same panel of the sequence and at the same time as the sequence at least two different spectral plots of the at least one spectrum showing two different product ions of at least one spectrum that support a cleavage of the at least one bond.

2. The method of claim 1, wherein the at least one spectrum is produced using electron capture dissociation (ECD).

3. The method of claim 1, wherein the sequence and the at least two different spectral plots are displayed in different subpanels of the same panel.

4. The method of claim 1, wherein the at least two different spectral plots are different zoomed-in portions of the at least one spectrum.

5. The method of claim 1, wherein the at least two different spectral plots further include theoretical product ion peaks or profiles of the two different product ions.

6. The method of claim 1, wherein the at least two different spectral plots further include a mass error of a product ion.

7. The method of claim 1, wherein the at least two different spectral plots further include a sequence of a product ion.

8. The method of claim 1, wherein the at least two different spectral plots further include different charge states of a product ion.

9. The method of claim 1, wherein the two different product ions comprise two complementary product ions of the sequence.

10. The method of claim 1, further comprising displaying an interactive icon between each two elements of the sequence if a product ion of the at least one spectrum is found to support a cleavage of a bond between the each two elements.

11. The method of claim 1, further comprising not displaying an interactive icon between each two elements of the sequence if no product ions of the at least one spectrum are found to support a cleavage of a bond between the each two elements.

12. The method of any combination of claim 1, further comprising calculating a count or score of products ions of the at least one spectrum that support a cleavage of a bond between each two elements of the sequence, producing a count or score of supporting product ions and charge states per bond position.

13. The method of claim 12, further comprising displaying an interactive icon between each two elements of the sequence that indicates the count or score if a product ion of the at least one spectrum is found to support a cleavage of a bond between the each two elements.

14. The method of claim 13, wherein a brightness of a color of the interactive icon indicates the count or score.

15. The method of claim 12, further comprising displaying on the display device in the same panel a plot of the count or score per bond position of the sequence.

16. A computer program product, comprising a non-transitory tangible computer-readable storage medium whose contents cause a processor to perform a method for providing a user interface for simultaneously displaying multiple pieces of spectral evidence linked to a bond of a polymeric compound, comprising:
providing a system, wherein the system comprises one or more distinct software modules, and wherein the distinct software modules comprise an input module and an analysis module;
receiving a polymeric compound sequence using the input module;
receiving at least one product ion spectrum known to include the polymeric compound using the input module, wherein one or more product ions of the at least one spectrum are assigned to at least one bond of the sequence;
displaying the sequence in a panel of a display device with at least one interactive icon between at least two elements of the sequence representing a bond of the sequence using the analysis module; and
when the at least one interactive icon is selected, displaying in the same panel of the sequence and at the same time as the sequence at least two different spectral plots showing two different product ions of the at least one spectrum that support a cleavage of the bond using the analysis module.

17. A system for providing a user interface for simultaneously displaying multiple pieces of spectral evidence linked to a bond of a polymeric compound, comprising:
a processor that
receives a polymeric compound sequence;
receives at least one product ion spectrum known to include the polymeric compound, wherein one or more product ions of the at least one spectrum are assigned to at least one bond of the sequence,
displays the sequence in a panel of a display device with at least one interactive icon between at least two elements of the sequence representing a bond of the sequence; and
when the at least one interactive icon is selected, displays in the same panel of the sequence and at the same time as the sequence at least two different spectral plots showing two different product ions of the at least one spectrum that support a cleavage of the bond.
